(19) United States
(12) Patent Application Publication
(10) Pub. No.: US 2010/0016660 A1
(43) Pub. Date: Jan. 21, 2010

(54) BENDING NECK FOR TRANSESOPHAGEAL ECHOCARDIOGRAPHY (TEE) PROBE

(75) Inventor: John Jack Merlo, Bedford, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/517,119

(22) PCT Filed: Dec. 3, 2007
(86) PCT No.: PCT/IB07/54900

§ 371 Date: Jun. 1, 2009

Related U.S. Application Data
(60) Provisional application No. 60/868,461, filed on Dec. 4, 2006.

(51) Int. Cl. A61B 1/00; B23K 26/00

(52) U.S. Cl. 600/104; 600/141; 219/121.72

(57) ABSTRACT

The invention provides articles of manufacture and methods for producing and using a bending neck assembly for a medical probe, using a tubular structure having a region of helical groove.

The technical field of this invention is systems and methods for producing and using a bending neck assembly for a medical probe.

Medical probes are widely used by physicians for a variety of functions, for example, for imaging, taking a biopsy sample, and/or operating on internal organs and tissues, while minimizing the size of incisions. One such type of probe is a transesophageal echocardiogram (TEE) probe, which is used to record an ultrasonic image of cardiac tissue. A distal tip of the probe, which features an ultrasonic transducer, is inserted into a patient's esophagus, where ultrasound waves are used to make an image of heart chambers, valves and surrounding structures. The TEE probe measures cardiac output and detects pericardial features such as inflammation or abnormalities of heart valves.

Additional types of medical probes include endoscopes and gastroscopes. Each probe is about 8 to 12 mm in diameter and about 150 cm in length, and is used for imaging, taking a biopsy sample, and/or operating on internal organs and tissues. These probes house control cables to maneuver a tip, glass fibers to transmit light from the tip, fiberoptic cable or electronic circuits to transmit an image back to an operator, and channels through which to pass instruments, air, suction, and water. Gastroscopes are primarily used within the gastrointestinal tract (e.g. stomach, small intestine) and are inserted orally into the esophagus, while the general term endoscope is applied to devices used in various parts of the body (e.g. respiratory tract, joints, or chest cavity) and may be inserted into a variety of body cavities.

A bending probe, such as a TEE probe or an endoscope, includes a bending neck assembly, which allows the probe to bend in order to acquire desired images or views or to navigate in curved passages. Currently, a bending neck assembly is made of many components or links. These components are manufactured from multiple tightly toleranced parts. Precision tooling and highly skilled production operators are needed to produce and assemble these components. Because of this complexity, bending neck assemblies are expensive to build.

Accordingly, an exemplary embodiment provided by the invention herein is a bending neck assembly for a probe for insertion and/or use in a bodily cavity, having: a tubular structure, for housing probe components and/or providing passage for probe components, the tubular structure having a flexible region of helical groove, in which the groove has a plurality of complete helical turns.

In a related embodiment of the assembly, the flexible region bends to an angle of at least 110°, or to an angle of at least 140°, or even to an angle of at least 170°. In another related embodiment, the flexible region bends in at least four planes relative to an axis of its length: anterior, posterior, medial, and lateral.

In another related embodiment, the assembly is composed of a metal, for example, at least one of the following types: titanium, stainless steel, beryllium copper, and phosphor bronze. In a preferred embodiment, the metal includes stainless steel.

In another related embodiment of the assembly, a length of the flexible region is at least about 0.5 inches to about 10.0 inches, or at least about 1.0 inches to about 20 inches, or about 4.0 inches to about 40 inches. In other embodiments, the flexible region is less than about 10 inches in length, or less than about 8 inches in length, or less than about 6 inches in length, or less than about 4 inches in length. In a related embodiment, the outer diameter of the assembly is at least about 0.1 inches to about 5.0 inches. In a related embodiment, the inner diameter of the assembly is at least about 0.1 inches to about 5.0 inches.

Another exemplary embodiment of the invention provided herein is a probe for insertion and/or use inside a bodily cavity, having: a distal tip portion containing a diagnostic and/or therapeutic component; a bending neck assembly continuous with the distal tip portion, in which the assembly provides a tubular structure, the tubular structure having a flexible region of helical groove, in which the groove has a plurality of complete helical turns; a handle portion continuous with the assembly and providing a controller; and a cover, continuous with the handle portion, extending to the distal tip portion, and covering an indwelling portion which includes the bending neck assembly, in which the indwelling portion is flexible in response to the controller.

In a related embodiment of the assembly, the diagnostic component includes an ultrasound transducer or other imaging component. In another related embodiment, the therapeutic component includes a surgical instrument.

Another exemplary embodiment is a method for using a bending neck assembly for a probe for insertion and/or use in a bodily cavity, the method having the steps of: incorporating the assembly into the probe, in which the assembly provides a tubular structure, having a flexible region of helical groove, such that the groove has a plurality of complete helical turns; inserting the probe into the bodily cavity; and using a controller to bend the probe, thereby imaging and/or manipulating an area or volume within the bodily cavity.

In a related embodiment of the above method, the probe further includes a transesophageal echocardiography probe or an endoscope. Further, the endoscope includes at least one of the following types: a sigmoidoscope, a gastroscope, a duodenoscope, an esophagoscope, a laryngoscope, a nasopharyngoscope, a rhinolaryngoscope, a cystoscope, an hysteroscope, a uteteroscope, a bronchoscope, a choledochoscope, a colonoscope, and an enteroscope.

Another exemplary embodiment provided by the invention herein is a method of making a bending neck assembly for a probe for insertion and/or use in a bodily cavity, including the steps of: producing a region of helical groove in a tubular structure, resulting in a flexible tubular structure, in which the tubular structure is for a probe for insertion and/or use in a bodily cavity.

A related embodiment of the above method further involves producing the region of helical groove by using a laser cutting technique or a machining technique.

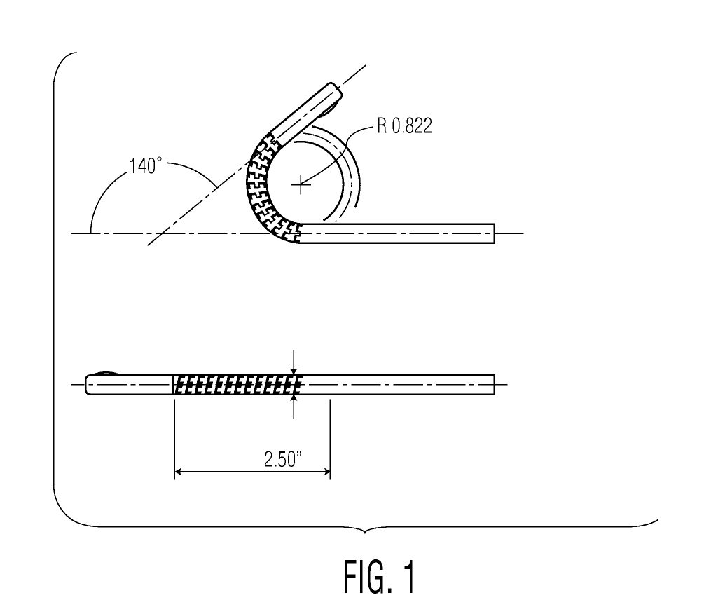

FIG. 1 shows a tubular structure, into which a helical groove has been cut, thereby providing a flexible tubular structure.

Currently, a bending neck assembly is made of many expensive components that require precision tooling and highly-skilled manufacturers. Producing a bending neck assembly from a tubular structure would greatly reduce the number of components involved in manufacture of a bending neck assembly, thereby simplifying a build process and reducing the level of skill required by the manufacturers. Simplifying the process would yield a cheaper, simpler and more reliable bending neck assembly.

A new type of bending neck assembly provided herein is used in manufacture of Transesophageal Echocardiography (TEE) probes and endoscopes that require flexibility.

An embodiment of the invention is shown in FIG. 1. A tubular structure has an outer diameter (designated as O.D. in FIG. 1) of about 0.1 inches to about 5.0 inches and has an inner diameter of about 0.1 inches to about 5.0 inches. A tubular structure, as used herein, includes one or more of the following types: a single-piece tubular object, two tubular objects having a press fit (also referred to as a co-extrusion), or a plurality of objects having a press fit.

A press fit (also referred to as an interference fit) is a fastening between two parts, which is achieved by friction after the parts are pushed together, rather than by any other means of fastening. For metal parts in particular, the friction that holds the parts together is often greatly increased by compression of one part against the other, which relies on the tensile and compressive strengths of the material the parts are made from.

The tubular structure is composed of metal, for instance titanium, stainless steel, beryllium copper, and/or phosphor bronze. The tubular structure has a shape, for instance, cylindrical or for instance, tapered on the interior, resulting in a tubular structure that is flexible in response to a controller.

A region of helical groove, for instance a continuous region of helical groove (designated as Helical Cuts in FIG. 1), is produced in the tubular structure, resulting in a flexible tubular structure. The groove as described herein encompasses any form of cut or indentation of any length, depth, or thickness. The helical groove has a plurality of, i.e. at least two, complete helical turns, i.e. each of which runs the entire circumference of the tubular structure, and is offset by the pitch of the helix. The helical groove is produced by means of a laser cutting technique or other machining technique.

The bending neck assembly is able to flex to an angle of at least about 140° (as shown in FIG. 1), for instance to an angle of at least about 170°, i.e. it can bend essentially to the extent of a U-shape, i.e. it can turn back on itself. The assembly also bends in at least four planes relative to its length: anterior, posterior, medial, and lateral (as indicated in FIG. 1).

A helical groove provides a flexible tubular structure, for instance providing a tubular structure for imaging, taking a biopsy sample, and/or otherwise manipulating an area or volume within a bodily cavity or to provide a probe for navigating in a passage within a bodily cavity which is not straight. The term “manipulate” as used herein refers to any form of producing real-time images or still images (e.g. photographs) of, touching, sampling, or operating on any area or volume within a bodily cavity.

A bodily cavity, as used herein, refers to any point, area, or volume within the interior of an animal body, for instance a mammal's body, for instance a human body or the body of an animal, for instance a high-value animal, for instance a cow or a horse. Cavities of the body include but are not limited to: an esophagus, a trachea, nasal passages, sinuses, a peritoneal cavity, fallopian tubes, vaginal passages, vas deferens, a rectum, and joints such as knees, elbows, or shoulders.

The bending neck assembly is one of many probe components. In a typical embodiment, a probe also includes: a distal tip portion, a proximal handle portion, and a cover.

The distal tip portion (designated as Ultrasound Window in FIG. 1) is located at the distal end of the probe and contains a diagnostic and/or therapeutic component, for instance an ultrasound transducer, a surgical instrument, or other type of imaging, sampling, or surgical component. A distinction between a diagnostic and a therapeutic component is difficult to make; for instance, a surgical instrument is used diagnostically for taking a biopsy sample and is also used therapeutically to remove tumorous tissue.

The bending neck assembly, which lies underneath a probe cover, is located along the axis of the probe's length between the distal tip portion and the handle portion. In some embodiments, the flexible portion of the assembly spans the entire length between the distal tip portion and the proximal handle portion. In other embodiments, inflexible portions are located between the flexible portion and the distal tip portion and/or between the flexible portion and the proximal handle portion.

The proximal handle portion includes a controller for controlling the flexible portion. The cover, for instance, made of plastic, for instance polyethylene, or made of metal, for instance stainless steel, covers an indwelling portion of the probe, including the bending neck assembly. The cover extends along the length of the probe from the distal tip portion to the proximal handle portion and protects the interior of the probe from the bodily cavity, and vice versa.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting.

What is claimed is:

1. A bending neck assembly for a probe for insertion and/or use in a bodily cavity, comprising: a tubular structure, housing probe components and/or providing passage for probe components, wherein the tubular structure comprises a flexible region of helical groove, wherein the groove has a plurality of complete helical turns.

2. The assembly according to claim 1, wherein the flexible region bends to an angle of at least 110°.

3. The assembly according to claim 1, wherein the flexible region bends to an angle of at least 140°.

4. The assembly according to claim 1, wherein the flexible region bends to an angle of at least 170°.

5. The assembly according to claim 1, wherein the flexible region bends in at least four planes relative to an axis of its length: anterior, posterior, medial, and lateral.

6. The assembly according to claim 1, wherein the assembly comprises a metal.

7. The assembly according to claim 6, wherein the metal comprises at least one selected from the group consisting of: titanium, stainless steel, beryllium copper, and phosphor bronze.

8. The assembly according to claim 6, wherein the metal comprises stainless steel.

9. The assembly according to claim 1, wherein a length of the flexible region is at least about 0.5 inches to about 10.0 inches.

10. The assembly according to claim 1, wherein the outer diameter of the assembly is at least about 0.1 inches to about 5.0 inches.

11. The assembly according to claim 1, wherein the inner diameter of the assembly is about 0.1 inches to about 5.0 inches.

12. A probe for insertion and use inside a bodily cavity, comprising: a distal tip portion containing a medical device; a bending neck assembly continuous with the distal tip portion, wherein the assembly comprises a tubular structure, having a flexible region of helical groove, wherein the groove has a plurality of complete helical turns; a handle portion comprising a controller; and a cover, continuous with the handle portion and covering an indwelling portion of the probe, wherein the indwelling portion is flexible in response to the controller.

13. The probe according to claim 12, wherein the diagnostic component comprises an ultrasound transducer and/or other imaging component.

14. The probe according to claim 12, wherein the therapeutic component comprises a surgical instrument.

15. The probe according to claim 12, wherein the indwelling portion of the probe comprises the bending neck assembly.

16. A method for using a bending neck assembly for a probe for insertion and/or use in a bodily cavity, comprising:
incorporating the assembly into the probe, wherein the assembly comprises a tubular structure having a flexible region of helical groove, wherein the groove has a plurality of complete helical turns;
inserting the probe into the bodily cavity; and
using a controller to bend the probe, thereby imaging and/or manipulating an area or volume within the bodily cavity.

17. The method according to claim 16, wherein the probe comprises a transesophageal echocardiography probe or an endoscope.

18. The method according to claim 17, wherein the endoscope comprises at least one selected from the group consisting of: a sigmoidoscope, a gastroscope, a duodenoscope, an esophagoscope, a laryngoscope, a nasopharyngoscope, a rhinolaryngoscope, a cystoscope, an hysteroscope, a uteteroscope, a bronchoscope, a choledochoscope, a colonoscope, and an enteroscope.

19. A method of manufacture of a bending neck assembly for a probe for insertion and/or use in a bodily cavity, the method comprising:
producing a helical groove in a tubular structure, thereby providing a flexible tubular structure, wherein the tubular structure is for a probe for insertion and/or use in a bodily cavity.

20. The method according to claim 19, wherein producing the helical groove further comprises using a laser cutting technique or a machining technique.
